(19) United States
(12) Patent Application Publication
(10) Pub. No.: US 2025/0017877 A1
(43) Pub. Date: Jan. 16, 2025

(54) PHYTONADIONE FOR PARENTERAL ADMINISTRATION

(71) Applicant: FRESENIUS KABI AUSTRIA GMBH, Graz (AT)

(72) Inventors: Patricia SEGER, Graz (AT); Theofanis MANTOURLIAS, Mainz (DE); Lorena MUGGETTI, Media (MB) (IT)

(73) Assignee: FRESENIUS KABI AUSTRIA GMBH, Graz (AT)

(21) Appl. No.: 18/705,423

(22) PCT Filed: Oct. 20, 2022
(86) PCT No.: PCT/EP2022/079171

§ 371 Date: Apr. 26, 2024

(30) Foreign Application Priority Data
Oct. 27, 2021 (EP) 21204947.2

(51) Int. Cl. A61K 31/122; A61K 47/12; A61K 47/44

(52) U.S. Cl. CPC A61K 31/122; A61K 47/12; A61K 47/44

(57) ABSTRACT

The present invention relates to an aqueous pharmaceutical composition for parenteral administration to a human, comprising a pharmacologically effective amount of a pharmaceutically acceptable vitamin K, a polyethoxylated castor oil as solubilizer, and a citrate buffer at a concentration of 1 to 40 mmol/L, wherein the pH of the composition is between 3.5 and 7.0, to a pharmaceutically acceptable container comprising the composition, and to the composition for use in the treatment or prevention of vitamin K deficiency and/or haemorrhage or for use as an antidote to anticoagulants of the coumarin type as well as to a method for preparing the composition.

### FIELD OF THE INVENTION

The present invention relates to a pharmaceutical composition for parenteral administration to a human, comprising a pharmacologically effective amount of a pharmaceutically acceptable vitamin K, to a pharmaceutically acceptable container comprising the pharmaceutical composition, to the pharmaceutical composition for use in the treatment or prevention of vitamin K deficiency and/or haemorrhage or for use as an antidote to anticoagulants of the coumarin type and to a method for preparing the pharmaceutical composition.

### BACKGROUND OF THE INVENTION

Vitamin K is a family of fat-soluble vitamers required in the body of humans and animals for post-translational modifications of proteins involved in blood coagulation. The vitamin K family is chemically derived from 2-methyl-1,4-naphthochinone and comprises two naturally occurring vitamers: vitamin K₁ (phytonadione or phylloquinone) and vitamin K₂ (menaquinone). Vitamin K₁ is used to treat certain bleeding disorders caused by vitamin K deficiency. A parenteral administration typically leads to beneficial effects within two hours.

Several commercial pharmaceutical vitamin K₁ preparations have been developed for use in humans as well as for the veterinary medicine market. AquaMEPHYTON (Telligent Pharma), Vitamin K-phytonadione injection (Hospira) and Phytonadione injectable emulsion (Dr. Reddy's) all represent injectable vitamin K₁ preparations sold in ampoules containing 2 mg/mL or 10 mg/mL phytonadione, 70 mg/mL polyoxyethylated fatty acid derivative, 37.5 mg/mL dextrose, 9 mg/mL benzyl alcohol in water at pH 5.0-7.0 (or 6.3), with hydrochloric acid or acetic acid optionally included to adjust pH. K-Ject (Henry Schein® Animal health) is a vitamin K₁ preparation for veterinary use sold in multiple dose vials containing 10 mg/mL phytonadione, 65 mg/mL polyoxyethylated fatty acid derivative, 37.5 mg/mL dextrose, 1 mg/mL butylated hydroxyanisole (BHA), 1 mg/mL butylated hydroxytoluene (BHT), 8.4 mg/mL citric acid, 17.2 mg/mL sodium phosphate, 9 mg/mL benzyl alcohol, in water at pH 5.0-7.0. All these formulations are either stored under reduced oxygen atmosphere, e.g. in ampoules, and/or contain synthetic antioxidants to avoid side reactions due to the presence of atmospheric oxygen resulting in reduced storage stability of the respective formulations. However, both options, avoidance of oxygen exposure and the presence of synthetic antioxidants, e.g., BHA and BHT, have considerable disadvantages. The need to work under the exclusion of oxygen requires complicated production processes. Antioxidants may lead to additional side reactions and/or are suspected of being harmful, which especially for parenteral administration to humans is considered critical.

Phytonadione injection, emulsion (International Medication Systems (IMS)) is a vitamin K₁ preparation sold in kits containing a SAF-T-Jet™ vial injector and a single dose vial containing 2 mg/mL phytonadione, 20 mg/mL polysorbate 80, 20.8 mg/mL propylene glycol, 0.34 mg/mL sodium acetate anhydrous, and 0.04 μL/mL glacial acetic acid in water, at pH 3.5-7.0. Administration of the IMS kit product to a human involves removing the protective caps from the vial and injector, aligning the vial such that the injector needle is centered on the stopper, threading the vial into the injector to allow the needle to penetrate the stopper, flipping the needle shield down, removing the needle cover, injecting the contents into the human, and reactivating the needle shield. The package insert for the IMS product includes several warnings, including that pushing the vial into the injector may cause misalignment of the injector needle and improper engaging may cause glass breakage and subsequent injury.

Hence, there is a need for pharmaceutical vitamin K₁ compositions for parenteral administration to humans in convenient containers easier to handle than ampoules or injection kits, and which are free of antioxidants.

### SUMMARY OF THE INVENTION

The inventors have surprisingly found that an aqueous pharmaceutical composition for parenteral administration to a human, comprising a pharmacologically effective amount of a pharmaceutically acceptable vitamin K, a polyethoxylated castor oil as solubilizer, and a citrate buffer at a concentration of 1 to 40 mmol/L, wherein the pH of the composition is between 3.5 and 7.0 has an excellent storage stability without a need of using antioxidants and without a need of excluding oxygen from the formulation. Consequently, the compositions according to the present invention provide for less complex formulations and for the reduction of side effects emanating from commonly used antioxidants. Furthermore, the compositions can be filled in convenient containers, e.g. glass vials, that may be handled easier than ampoules or other containers that allow for a rigorous exclusion of oxygen. This allows for commercially attractive (i.e. cost effective) production processes as well as for convenient handling of the compositions.

Accordingly, in one aspect of the invention an aqueous pharmaceutical composition for parenteral administration to a human is provided, wherein the composition comprises a pharmacologically effective amount of a pharmaceutically acceptable vitamin K, a polyethoxylated castor oil as solubilizer, and a citrate buffer at a concentration of 1 to 40 mmol/L, wherein the pH of the composition is between 3.5 and 7.0.

In another aspect the present invention relates to a pharmaceutically acceptable container comprising an aqueous pharmaceutical composition for parenteral administration to a human, wherein the composition comprises a pharmacologically effective amount of a pharmaceutically acceptable vitamin K, a polyethoxylated castor oil as solubilizer, and a citrate buffer at a concentration of 1 to 40 mmol/L, wherein the pH of the composition is between 3.5 and 7.0 and wherein the container is not an ampoule.

In yet another aspect the present invention relates to an aqueous pharmaceutical composition for parenteral administration to a human, wherein the composition comprises a pharmacologically effective amount of a pharmaceutically acceptable vitamin K, a polyethoxylated castor oil as solubilizer, and a citrate buffer at a concentration of 1 to 40 mmol/L, wherein the pH of the composition is between 3.5 and 7.0 for use in the treatment or prevention of vitamin K deficiency and/or haemorrhage or for use as an antidote to anticoagulants of the coumarin type.

In a further aspect the present invention relates to a method for preparing an aqueous pharmaceutical composition for parenteral administration to a human comprising a pharmacologically effective amount of a pharmaceutically acceptable vitamin K, a polyethoxylated castor oil as solubilizer, and a citrate buffer at a concentration of 1 to 40 mmol/L, wherein the pH of the composition is between 3.5 and 7.0, the method comprising the steps of
a) obtaining a mixture of a polyethoxylated castor oil and vitamin K by adding vitamin K to the polyethoxylated castor oil under stirring,
b) heating the mixture of step a) preferably to a temperature of 60-65° C. under stirring,
c) heating water for injection (WFI) preferably to 60-65° C. and optionally purging the heated water with nitrogen,
d) obtaining a dispersion by adding the WFI of step c) to the mixture of step b) under stirring, wherein the temperature is kept preferably in the range of 60-65° C.,
e) cooling the dispersion obtained in step d) to preferably 25° C. under stirring,
f) adding a pharmaceutically acceptable tonicity agent, preservative, and buffer to the dispersion of step e) and stirring until the tonicity agent, preservative, and buffer have completely dissolved,
g) adding at least one agent to adjust the pH of the dispersion of step f) to between 3.5 to 7, preferably 5.0 to 7.0, more preferably to 6.0 to 6.5,
h) adding WFI to the dispersion of step g) to adjust the final volume of the dispersion,
i) filtering the dispersion of step h) through a pharmaceutically acceptable filter, preferably a filter having a pore size of 0.22 μm,
j) optionally sterilizing the dispersion of step i).

### DETAILED DESCRIPTION OF THE INVENTION

### Definitions

“Vitamin K” as used herein refers to vitamin K₁, vitamin K₂ and vitamin K₃. Phytonadione, phyllochinone or phytomenadione as used herein are synonyms for vitamin K₁.

“Dextrose” as used herein refers to D-(+)-dextrose as well as to hydrates thereof, such as e.g. D-(+)-dextrose monohydrate.

“Kolliphor™ EL” as used herein refers to a non-ionic solubilizer made by reacting castor oil with ethylene oxide in a molar ratio of 1:35. Kolliphor™ EL is also known under the generic names macrogolglycerol ricinoleate polyoxyl-35-castor oil or PEG-35 castor oil, respectively.

“Kolliphor™ ELP” as used herein refers to a highly purified version of Kolliphor™ EL for sensitive active ingredients.

“Citrate” as used herein refers to citric acid and its mono-, di- and tri-deprotonated anions as well as to pharmaceutically acceptable salts and hydrates thereof.

The citrate buffer concentration herein refers to the sum of the concentration of citric acid, its mono-, di- and tri-deprotonated anions and its pharmaceutically acceptable salts and hydrates.

“Phosphate” as used herein refers to phosphoric acid, dihydrogenphosphate, hydrogenphosphate and phosphate anions as well as to pharmaceutically acceptable salts and hydrates thereof.

The phosphate buffer concentration herein refers to the sum of the concentration of phosphoric acid, dihydrogenphosphate, hydrogenphosphate, phosphate and its pharmaceutically acceptable salts and hydrates.

“Histidine” as used herein refers to histidine and its mono-, di-, and tri-protonated cations as well as to pharmaceutically acceptable salts and hydrates thereof.

The histidine buffer concentration herein refers to the sum of histidine and its mono-, di-, and tri-protonated cations and its pharmaceutically acceptable salts and hydrates.

“mmol/L” or mM as used herein refers to mmol per liter of the pharmaceutical composition.

“Butylated hydroxyanisole” (BHA) as used herein refers to an antioxidant consisting of a mixture of two isomeric organic compounds, 2-tert-butyl-4-hydroxyanisole and 3-tert-butyl-4-hydroxyanisole.

“Butylated hydroxytoluene” (BHT), refers to the antioxidant 2,6-Di-tert-butyl-4-methylphenol, also known as dibutylhydroxytoluene.

“Substituted phenols” as used herein refers to chemically modified phenols, such as alkylated phenols or alkylated phenolether, which have antioxidant properties. BHA and BHT are examples of substituted phenols.

“PVDF” as used herein refers to Polyvinylidendifluorid.

### The Pharmaceutical Composition

The aqueous pharmaceutical composition for parenteral administration to a human according to the present invention comprises a pharmacologically effective amount of a pharmaceutically acceptable vitamin K, a polyethoxylated castor oil as solubilizer, and citrate buffer at a concentration of 1 to 40 mmol/L. The pH of the composition is between 3.5 and 7.0, preferably between 5.0 and 7.0, more preferably between 6.0 and 6.5, most preferably the pH of the composition is 6.3. Pharmaceutically acceptable forms of vitamin K as well as their pharmacologically effective amounts are known to the person skilled in the art.

In the vitamin K formulations according to the present invention the citrate buffer is present at a concentration of 1 to 40 mmol/L. In contrast to other buffer systems investigated—citrate buffer surprisingly resulted in sufficiently stable vitamin K compositions without a need to use antioxidants and without a need to work under exclusion of oxygen. Thus, the major drawbacks of certain commercial vitamin K preparations—the need to use sealed ampoules to avoid oxygen exposure and/or the use of antioxidants—are overcome by the compositions of the present invention. In a preferred embodiment, the concentration of the citrate buffer may be in the range of 1 to 20 mmol/L, preferably 1 to 10 mmol/L, more preferably 2 to 6 mmol/L, even more preferably 4.5 to 5.5 mmol/L, most preferably 5 mmol/L.

It is contemplated that other buffers may be suitable for use in the vitamin K formulations according to the present invention. In some embodiments, the buffer is a weak acid and a conjugate base of the weak acid. In certain embodiments, the weak acid is a dicarboxylic acid or a tricarboxylic acid. In some embodiments, the acid is citric acid, isocitric acid, aconitic acid, trimesic acid, propane-1,2,3-tricarboxylic acid, fumaric acid, oxalic acid, maleic acid, malonic acid, glutaric acid, succinic acid or tartaric acid. In some embodiments, the concentration of the buffer may be in the rage of 0.5 to 50 mmol/L, preferably 1 to 20 mmol/L, more preferably 1 to 10 mmol/L, more preferably 2 to 6 mmol/L, even more preferably 4.5 to 5.5 mmol/L, most preferably 5 mmol/L.

The pH of the formulation of the invention is stable following storage under room temperature and/or accelerated conditions. Preferably, the pH drift of the formulation is less than 0.3 pH units, e.g., less than 0.25 pH units, less than 0.2 pH units, less than 0.15 pH units, less than 0.1 pH units, or less than 0.05 pH units following storage for at least 6 months, at least 12 months, or at least 24 months at room temperature and/or accelerated conditions.

The composition according to the present invention comprises a polyethoxylated castor oil as a solubilizer. In a preferred embodiment, the polyethoxylated castor oil is obtained by reacting castor oil with ethylene oxide in a molar ratio of 1:35. In a more preferred embodiment, the polyethoxylated castor oil is Kolliphor™ EL or Kolliphor™ ELP. In a particularly preferred embodiment, the polyethoxylated castor oil is Kolliphor™ ELP. The concentration of the polyethoxylated castor oil, preferably the concentration of Kolliphor™ ELP or Kolliphor™ EL, may be 30 to 150 mg per mL of the composition, preferably 50 to 100 mg per mL of the composition, more preferably 60 to 80 mg per mL of the composition, most preferably 70 mg per mL of the composition.

The composition according to the present invention comprises a pharmacologically effective amount of a pharmaceutically acceptable vitamin K. Preferably, the pharmaceutically acceptable vitamin K is vitamin K₁ (phytonadione). Preferably, the composition of the present invention comprises 0.1 to 50 mg per mL, more preferably 0.5 to 20 mg per mL, most preferably 1 to 11 mg vitamin K₁ (phytonadione) per mL of the composition. In particularly preferred embodiments the vitamin K₁ (phytonadione) concentration in the composition is 2 mg or 10 mg per mL of the composition.

In a preferred embodiment, the composition of the present invention does not comprise butylated hydroxxyanisole (BHA) or butylated hydroxytoluene (BHT). In a more preferred embodiment, the composition of the present invention does not comprise an antioxidant from the group of substituted phenols. In an even more preferred embodiment, the composition of the present invention does not comprise an antioxidant. The inventors surprisingly found that the compositions according to the present invention have sufficient storage stability in the absence of antioxidants—making the compositions less complex and reducing side effects and/or toxicities emerging from the active ingredient and/or excipients.

In one embodiment, the composition of the present invention comprises a pharmaceutically acceptable tonicity agent. The person skilled in the art is aware of suitable pharmaceutically acceptable tonicity agents. Preferably, the pharmaceutically acceptable tonicity agent is selected from the group consisting of dextrose, glycerol, mannitol, sodium chloride, and mixtures thereof. More preferably, the pharmaceutically acceptable tonicity agent is dextrose, most preferably dextrose monohydrate. The composition of the present invention may comprise 30 to 45 mg, preferably 35 to 40 mg, more preferably 37.5 mg dextrose monohydrate per mL of the composition.

The composition according to the present invention may, in a further embodiment, comprise a pharmaceutically acceptable preservative. The person skilled in the art is aware of pharmaceutically acceptable preservatives. Preferably, the pharmaceutically acceptable preservative is benzyl alcohol. Preferably, the concentration of benzyl alcohol is 1 to 20 mg, preferably 5 to 15 mg, more preferably 8 to 10 mg benzyl alcohol per mL of the composition.

The pharmaceutical composition according to the present invention may comprise at least one agent for adjusting the pH of the composition. The at least one agent for adjusting the pH of the composition is preferably selected from the group consisting of glacial acetic acid, hydrochloric acid, and sodium hydroxide.

In one embodiment, the pharmaceutical composition according to the present invention comprises phytonadione, citrate buffer at a concentration of 1 to 40 mmol/L, dextrose, benzyl alcohol, 30 to 150 mg Kolliphor™ ELP per mL of the composition, water for injection and glacial acetic acid and has a pH between 6.0 and 6.5.

In a preferred embodiment, the pharmaceutical composition comprises 2 mg phytonadione per mL of the composition, 5 mmol citrate buffer per liter of the composition, 37.5 mg dextrose monohydrate per mL of the composition, 9 mg benzyl alcohol per mL of the composition, 70 mg Kolliphor™ ELP per mL of the composition, water for injection and glacial acetic acid and has a pH of 6.3.

In another preferred embodiment, the pharmaceutical composition comprises 10 mg phytonadione per mL of the composition, 5 mmol citrate buffer per liter of the composition, 37.5 mg dextrose monohydrate per mL of the composition, 9 mg benzyl alcohol per mL of the composition, 70 mg Kolliphor™ ELP per mL of the composition, water for injection and glacial acetic acid and has a pH of 6.3.

### Stability of the Pharmaceutical Composition

The pharmaceutical compositions according to the present invention are storage stable. As used herein, the terms “stable” and “stability” encompass any characteristic of the formulation which may be affected by storage conditions including, without limitation, potency, total impurities, phytonadione degradation products, headspace oxygen, specific optical rotation, optical purity, appearance, viscosity, sterility, particulates (visible and subvisible), and color and clarity. The storage conditions which may affect stability include, for example, duration, temperature, humidity, and/or light exposure.

In some embodiments, a stable formulation refers to a formulation that contains at least 95%, or at least 96%, or at least 97%, or at least 98%, or at least 98.8% of the theoretical amount of a pharmaceutically acceptable vitamin K after storage under room temperature (e.g., 25° C.±2° C./60% relative humidity (RH)±5% RH) and/or accelerated (e.g., at 40° C.±2° C./75% RH±5% RH) conditions. In other embodiments, a stable formulation refers to a formulation that contains less than 105%, or less than 103%, or less than 102%, of the of the theoretical amount of a pharmaceutically acceptable vitamin K after storage under room temperature and/or accelerated conditions. In some embodiments, a stable formulation refers to a formulation that contains 95% to 105%, preferably 97% to 103%, more preferably 98% to 102%, most preferably 98.8% to 102% of the theoretical amount of a pharmaceutically acceptable vitamin K after storage under room temperature and/or accelerated conditions.

In other embodiments, a stable formulation refers to a formulation that contains less than 5% (area percent), or less than about 4% (area percent), preferably less than 3% (area percent), more preferably less than 2% (area percent), most preferably less than 1.2% (area percent) of total vitamin K-related impurities present in the formulation after storage under room temperature and/or accelerated conditions.

In some embodiments, the vitamin K formulation of the invention is stable for at least 12 months, at least 18 months, at least 24 months, or at least 36 months at room temperature (e.g., at 25° C.±2° C./60% RH±5% RH). In other embodiments, the vitamin K formulation of the invention is stable for at least 1 month, at least 3 months, at least 6 months, or at least 12 months under accelerated conditions (e.g., at 40° C.±2° C./75% RH±5% RH).

The phytonadione content (i.e., API assay), individual impurities, and total impurities in the pharmaceutical compositions according to the present invention are measured using high-performance liquid chromatography (HPLC). In some embodiments, the HPLC is performed using an Agilent 1100/1200 equipped with a diode array detector (DAD) and a DAD wavelength of 330±4 nm and a reference wavelength of 600 nm±50 nm using a 60 mm DAD cell. In a preferred embodiment, an Aeris Peptide XB-C18 250 4.6 mm; 2.6 μm column and a mobile phase consisting of a mixture of 0.1% formic acid (A) and methanol (B) is used. In more preferred embodiment, a gradient is used (0.00-1.50 min: 10% A, 90% B, 1.50-25.00 min: 100% B, 25.01-32 min: 10% A, 90% B).

### The Container

The pharmaceutical composition according to the present invention is contained within a pharmaceutically acceptable container. According to the present invention the container is not an ampoule.

In one embodiment, the container is a vial sealed with a stopper, a syringe, or a cartridge. Preferably, the container is a glass vial, more preferably a light protective glass vial, most preferably an amber glass vial. In a preferred embodiment the container is an ammonium sulfate treated glass vial. The pharmaceutical composition of the present invention has excellent storage stability when stored in glass vials, e.g. in ammonium sulfate treated amber glass vials. In some embodiments, the vial is not a component of a kit comprising an injection device into which the vial is intended to be inserted prior to administration of the contents of the vial into a patient.

Where the container is a glass vial sealed with a stopper, the stopper material may comprise a rubber, preferably a halogenated isobutylene-isoprene copolymer, more preferably a chlorobutyl rubber.

Preferably, the stopper is coated. More preferably, the coating of the stopper comprises ethylene tetrafluoroethylene. Such coated stoppers do not interact with the pharmaceutical composition of the present invention, thereby increasing the storage stability of the composition. Coatings comprising ethylene tetrafluoroethylene are commercially available under the tradename FluroTec®.

In one embodiment, the stopper comprises a septum. A septum allows for an easy and convenient withdrawal of the pharmaceutical composition contained in the vial, by e.g. a syringe equipped with a needle without exposing the composition to the environment thereby reducing the risk of contamination and unwanted reactions with e.g. oxygen. Furthermore, the septum allows for a straightforward administration of the composition according to the present invention to a patient without the need to remove the stopper from the vial.

In some embodiments, the oxygen content in the headspace of the vials is greater than 1.0 vol %, e.g., greater than 1.5 vol % based on the total gas volume in the container. Preferably, the oxygen content in the headspace of the vials is 1.0-2.5%. Still, the inventors surprisingly found that the compositions according to the present invention are storage stable in vials. The compositions according to the present invention allow for a more straightforward fill and finishing process without the need to apply measures for a stringent elimination of oxygen. Thus, the manufacturing process is less complex and hence more cost efficient.

### Medical Use of the Pharmaceutical Composition

The present invention also relates to the use of the pharmaceutical composition according to the present invention for the treatment or prevention of vitamin K deficiency and/or haemorrhage or as an antidote to anticoagulants of the coumarin type. The person skilled in the art is aware of the respective dosages and dosing regimen.

### Method of Treating Vitamin K Deficiency

The present invention also relates to a method of treating or preventing a condition associated with vitamin K deficiency in a subject in need thereof, comprising administering a composition according to the present invention to the subject, thereby treating or preventing the condition in the subject.

Preferably, the condition is selected from the group consisting of (1) anticoagulant-induced prothrombin deficiency caused by coumarin or indanedione derivatives, (2) hemorrhagic disease of a newborn, (3) hypoprothrombinemia due to antibacterial therapy, (4) hypoprothrombinemia secondary to factors limiting absorption or synthesis of Vitamin K, and (5) other drug-induced hypoprothrombinemia where it is definitely shown that the result is due to interference with phytonadione injectable emulsion metabolism.

### Preparation of the Pharmaceutical Composition

The composition according to the present invention may be prepared by a method comprising the following steps:
a) obtaining a mixture of a polyethoxylated castor oil and vitamin K by adding vitamin K to the polyethoxylated castor oil under stirring,
b) heating the mixture of step a) preferably to a temperature of 60-65° C. under stirring,
c) heating water for injection (WFI) preferably to 60-65° C. and optionally purging the heated water with nitrogen,
d) obtaining a dispersion by adding the WFI of step c) to the mixture of step b) under stirring, wherein the temperature is kept preferably in the range of 60-65° C.,
e) cooling the dispersion obtained in step d) to preferably 25° C. under stirring,
f) adding a pharmaceutically acceptable tonicity agent, preservative, and buffer to the dispersion of step e) and stirring until the tonicity agent, preservative, and buffer have completely dissolved,
g) adding at least one agent to adjust the pH of the dispersion of step f) to between 3.5 to 7, preferably 5.0 to 7.0, more preferably to 6.0 to 6.5,
h) adding WFI to the dispersion of step g) to adjust the final volume of the dispersion,
i) filtering the dispersion of step h) through a pharmaceutically acceptable filter, preferably a filter having a pore size of 0.22 μm,
j) optionally sterilizing the dispersion of step i).

In a preferred embodiment all process steps are conducted under protection from oxygen, more preferably under nitrogen atmosphere. In another preferred embodiment all process steps are performed under protection from light, more preferably the process is conducted in a dimmed area using light with a wavelength >520 nm. According to a further embodiment of the present invention, all process steps are conducted under simultaneous protection from oxygen, such as under nitrogen atmosphere, and under protection from light, such as in a dimmed area using light with a wavelength >520 nm.

The sterilization according to step I) may be conducted via autoclaving. Preferably, the autoclaving takes place for 12-18 minutes at a temperature of 120-125° C. ° C. and a pressure of 2500-3500 mbar or by sterile filtration of the dispersion into a pre-sterilized container using e.g. a hydrophilic PVDF-filter with a pore size of 0.2 μm. The skilled person is aware of suitable membrane materials.

### Embodiments

1) Aqueous pharmaceutical composition for parenteral administration to a human, comprising a pharmacologically effective amount of a pharmaceutically acceptable vitamin K, a polyethoxylated castor oil as solubilizer, and citrate buffer at a concentration of 1 to 40 mmol/L, wherein the pH of the composition is between 3.5 and 7.0.

2) Composition according to embodiment 1, wherein the composition comprises citrate buffer at a concentration of 1 to 20 mmol/L, preferably 1 to 10 mmol/L, more preferably 2 to 6 mmol/L, even more preferably 4.5 to 5.5 mmol/L, most preferably 5 mmol/L.

3) Composition according to embodiment 1 or 2, wherein the pH of the composition is between 5.0 and 7.0, preferably between 5.5 and 6.5, more preferably between 6.0 and 6.5.

4) Composition according to any of the preceding embodiments, wherein the polyethoxylated castor oil is obtained by reacting castor oil with ethylene oxide in a molar ratio of 1:35.

5) Composition according to any of the preceding embodiments, wherein the polyethoxylated castor oil is Kolliphor™ ELP or Kolliphor™ EL.

6) Composition according to embodiment 5, comprising 30 to 150 mg, preferably 50 to 100 mg, more preferably 60 to 80 mg, most preferably 70 mg Kolliphor™ ELP or Kolliphor™ EL per mL of the composition.

7) Composition according to any of the preceding embodiments, wherein the pharmaceutically acceptable vitamin K is vitamin K₁ (phytonadione).

8) Composition according to embodiment 7 comprising 0.1 to 50 mg, preferably 0.5 to 20 mg, more preferably 1 to 11 mg phytonadione per mL of the composition.

9) Composition according to embodiment 8 comprising 2 mg phytonadione per mL of the composition.

10) Composition according to embodiment 8 comprising 10 mg phytonadione per mL of the composition.

11) Composition according to any of the preceding embodiments, wherein the composition does not comprise butylated hydroxyanisole (BHA) or butylated hydroxytoluene (BHT).

12) Composition according to any of the preceding embodiments, wherein the composition does not comprise an antioxidant from the group of substituted phenols.

13) Composition according to any of the preceding embodiments, wherein the composition does not comprise an antioxidant.

14) Composition according to any of the preceding embodiments further comprising a pharmaceutically acceptable tonicity agent.

15) Composition according to embodiment 14, wherein the pharmaceutically acceptable tonicity agent is selected from the group consisting of dextrose, glycerol, mannitol, sodium chloride, and mixtures thereof.

16) Composition according to embodiment 14 or 15, wherein the pharmaceutically acceptable tonicity agent is dextrose, preferably dextrose monohydrate.

17) Composition according to embodiment 16 comprising 30 to 45 mg, preferably 35 to 40 mg, more preferably 37.5 mg dextrose monohydrate per mL of the composition.

18) Composition according to any of the preceding embodiments further comprising a pharmaceutically acceptable preservative.

19) Composition according to embodiment 18, wherein the pharmaceutically acceptable preservative is benzyl alcohol.

20) Composition according to embodiment 19, comprising 1 to 20 mg, preferably 5 to 15 mg, more preferably 8 to 10 mg benzyl alcohol per mL of the composition.

21) Pharmaceutical composition according to any of the preceding embodiments comprising at least one agent for adjusting the pH of the composition.

22) Pharmaceutical composition according to embodiment 21, wherein the at least one agent for adjusting the pH of the composition is selected from the group consisting of glacial acetic acid, hydrochloric acid, and sodium hydroxide.

23) Pharmaceutical composition according to any of the preceding embodiments, comprising phytonadione, citrate buffer at a concentration of 1 to 40 mmol/L, dextrose, benzyl alcohol, Kolliphor™ ELP, water for injection and glacial acetic acid, wherein the pharmaceutical composition has a pH between 6.0 and 6.5.

24) Pharmaceutical composition according to any of the preceding embodiments, comprising phytonadione, citrate buffer at a concentration of 1 to 40 mmol/L, dextrose, benzyl alcohol, 30 to 150 mg Kolliphor™ ELP per mL of the composition, water for injection and glacial acetic acid, wherein the pharmaceutical composition has a pH between 6.0 and 6.5.

25) Pharmaceutical composition according to any of embodiments 1 to 9 and 11 to 24, comprising 2 mg phytonadione per mL of the composition, 5 mmol citrate buffer per liter of the composition, 37.5 mg dextrose monohydrate per mL of the composition, 9 mg benzyl alcohol per mL of the composition, 70 mg Kolliphor™ ELP per mL of the composition, water for injection and glacial acetic acid, wherein the pharmaceutical composition has a pH of 6.3.

26) Pharmaceutical composition according to any of embodiments 1 to 8 and 10 to 24, comprising 10 mg phytonadione per mL of the composition, 5 mmol citrate buffer per liter of the composition, 37.5 mg dextrose monohydrate per mL of the composition, 9 mg benzyl alcohol per mL of the composition, 70 mg Kolliphor™ ELP per mL of the composition, water for injection and glacial acetic acid, wherein the pharmaceutical composition has a pH of 6.3.

27) Pharmaceutically acceptable container comprising a pharmaceutical composition according to any of the preceding embodiments, wherein the container is not an ampoule.

28) Pharmaceutically acceptable container according to embodiment 27, wherein the pharmaceutically acceptable container is a vial sealed with a stopper, a syringe or a cartridge.

29) Pharmaceutically acceptable container according to embodiment 28, wherein the container is a vial sealed with a stopper and wherein the stopper material comprises a rubber, preferably a halogenated isobutylene-isoprene copolymer, more preferably a chlorobutyl rubber.

30) Pharmaceutically acceptable container according to embodiment 28 or 29, wherein the stopper is coated.

31) Pharmaceutically acceptable container according to embodiment 30, wherein the coating of the stopper comprises ethylene tetrafluoroethylene.

32) Pharmaceutically acceptable container according to embodiments 28 to 31, wherein the stopper comprises a septum.

33) Pharmaceutically acceptable container according to any of embodiments 28 to 32, wherein the vial is a glass vial, preferably a light protective glass vial, more preferably an amber glass vial.

34) Pharmaceutically acceptable container according to embodiment 33, wherein the glass is an ammonium sulfate treated glass.

35) Composition according to any of embodiments 1 to 26 for use in the treatment or prevention of vitamin K deficiency and/or haemorrhage or for use as an antidote to anticoagulants of the coumarin type.

36) Method of treating or preventing a condition associated with vitamin K deficiency in a subject in need thereof, comprising administering a composition according to any of embodiments 1 to 26 to the subject, thereby treating or preventing the condition in the subject.

37) Method according to embodiment 36, wherein the condition is selected from the group consisting of (1) anticoagulant-induced prothrombin deficiency caused by coumarin or indanedione derivatives, (2) hemorrhagic disease of a newborn, (3) hypoprothrombinemia due to antibacterial therapy, (4) hypoprothrombinemia secondary to factors limiting absorption or synthesis of Vitamin K, and (5) other drug-induced hypoprothrombinemia where it is definitely shown that the result is due to interference with phytonadione injectable emulsion metabolism.

38) Method for preparing a composition according to any of embodiments 1 to 26, comprising the steps of
a) obtaining a mixture of a polyethoxylated castor oil and vitamin K by adding vitamin K to the polyethoxylated castor oil under stirring,
b) heating the mixture of step a) preferably to a temperature of 60-65° C. under stirring,
c) heating water for injection (WFI) preferably to 60-65° C. and optionally purging the heated water with nitrogen,
d) obtaining a dispersion by adding the WFI of step c) to the mixture of step b) under stirring, wherein the temperature is kept preferably in the range of 60-65° C.,
e) cooling the dispersion obtained in step d) to preferably 25° C. under stirring,
f) adding a pharmaceutically acceptable tonicity agent, preservative, and buffer to the dispersion of step e) and stirring until the tonicity agent, preservative, and buffer have completely dissolved,
g) adding at least one agent to adjust the pH of the dispersion of step f) to between 3.5 to 7, preferably 5.0 to 7.0, more preferably to 6.0 to 6.5,
h) adding WFI to the dispersion of step g) to adjust the final volume of the dispersion,
i) filtering the dispersion of step h) through a pharmaceutically acceptable filter, preferably a filter having a pore size of 0.22 μm,
j) optionally sterilizing the dispersion of step i).

39) Method according to embodiment 38, wherein all process steps are conducted under protection from oxygen, preferably under nitrogen atmosphere.

40) Method according to embodiment 38 or 39, wherein all process steps are performed under protection from light.

41) Method according to any of embodiments 38 to 40, wherein in step j) the dispersion is sterilized by autoclaving, preferably for 12-18 minutes at a temperature of 120-125° C. ° C. and a pressure of 2500-3500 mbar.

42) Method according to any of embodiments 38 to 40, wherein in step j) the dispersion is sterilized by sterile filtration of the dispersion into a pre-sterilized container using e.g. a hydrophilic PVDF-filter with a pore size of 0.2 μm. examples

### EXAMPLES

### Analytical Methods

The following analytical methods were used to analyze the samples included in the stability studies of the following examples.

Determination of pH-Values

PH-values were measured potentiometrically (pH lab 827, Metrohm, equipped with a LL Biotrode 3 mm).

### Headspace Oxygen

Headspace oxygen was measured using an FMS-Oxygen Headspace Analyzer (Lighthouse) equipped with a near-infrared diode laser designed to measure oxygen concentration in sealed parenteral containers.

### Phytonadione Content/Total Impurities

Both parameters were measured using an HPLC-method employing an Agilent 1100/1200 equipped with a DAD 60 mm cell (DAD wavelength 330±4 nm; ref. 600 nm±50 nm). An Aeris Peptide XB-C18 250 4.6 mm; 2.6 μm column was used as stationary phase together with a mixture of 0.1% formic acid and methanol as mobile phase.

All sample preparation steps were done under strict light protection. Samples were filled in brown glass sample containers.

### Example 1: Preparation of Phytonadione Dispersions

The dispersions were prepared with an overall batch-size of 500 mL, corresponding to 5000 mg phytonadione per batch at a Phytonadione content of 10 mg/mL of the final dispersion. All operations took place under protection from light and oxygen in a dimmed area using yellow light (>520 nm).

A mixture of polyethoxylated castor oil (Kolliphor™ ELP or Kolliphor™ EL, 70 mg/mL of the final dispersion) and phytonadione (2 mg/mL or 10 mg/mL of the final dispersion) was prepared by adding phytonadione to the polyethoxylated castor oil under stirring in a glass vessel. The mixture was heated to 60-65° C. Water for injection (WFI), preheated to 60-65° C. and purged with nitrogen, was added to the polyethoxylated castor oil/phytonadione mixture under stirring at 60-65° C. The resulting dispersion was cooled to 25° C. under continuous stirring. Dextrose monohydrate (37.5 mg/mL of the final dispersion) was added and completely dissolved under stirring. Subsequently, benzyl alcohol (9 mg/mL of the final dispersion) was added and completely dissolved under stirring.

Five Different Buffer Systems were Investigated:
1. Citrate buffer pH=6.3 at a concentration of 5 mmol/L citrate (example 2 and 3), by adding citric acid monohydrate (0.147 mg/mL of the final dispersion) and sodium citrate dihydrate (1.265 mg/mL of the final dispersion).
2. Citrate buffer pH=6.3 at a concentration of 2.5 mmol/L citrate (example 2), by adding citric acid monohydrate (0.0735 mg/mL of the final dispersion) and sodium citrate dihydrate (0.6325 mg/mL of the final dispersion).
3. Citrate buffer pH=6.3 at a concentration of 1.0 mmol/L citrate (example 2), by adding citric acid monohydrate (0.0294 mg/mL of the final dispersion) and sodium citrate dihydrate (0.253 mg/mL of the final dispersion).
4. Phosphate buffer pH=6.3 at a concentration of 5 mmol/L phosphate (example 3), by adding Monosodium phosphate (0.516 mg/mL of the final dispersion) and disodium phosphate (0.099 mg/mL of the final dispersion).
5. Histidine buffer pH=6.3 at a concentration of 5 mmol/L histidine (example 3), by adding L-Histidine base (0.517 mg/mL of the final dispersion) and L-Histidine hydrochloride monohydrate (0.350 mg/mL of the final dispersion).

The respective buffered dispersions were stirred after the addition of the buffer salts until complete dissolution. The pH was adjusted to 6.3 by adding glacial acetic acid and WFI was added to adjust the final volume. The dispersions were filtered using a hydrophilic PVDF-filter with a pore size of 0.2 μm.

### Example 2: Stability Studies in Citrate Buffered Dispersions

The dispersions according to example 1 were incubated for 12 hours in a stainless-steel tank to study the effect of Fe³⁺ ions on the stability of the samples. Aliquots were filled into ammonium sulfate treated amber 2 cc vials and closed with 13 mm Flurotec™ coated chlorobutyl stoppers. The headspace oxygen content of the samples was 2%.

These samples were stored at 25° C. and 40° C. for up to 12 months (M) in stability chambers. Vials were stored upside-down (to ensure stopper contact).

### Reference Batches:

Additionally, two batches of a commercial phytonadione formulation (Vitamin K1-phytonadione injection in ampoules, Hospira), were stored as reference samples:
Reference sample 1 (Phytonadione content=2 mg/mL); 19 months into shelf-life at “T=0” of the stability studies
Reference sample 2 ((Phytonadione content=10 mg/mL); 14 months into shelf-life at “T=0” of the stability studies.

The reference samples were stored in their original container (i.e. ampoules with a headspace oxygen content below 1%) during the stability studies.

PH-values, headspace oxygen and phytonadione content and total impurities were measured using the analytical methods described above.

### Results:

pH-Values

TABLE 2a
pH-values of stability samples

| Phytonadione | | | 25° C./3 | 25° C./6 | 25° C./12 | 40° C./3 | 40° C./6 |
|---|---|---|---|---|---|---|---|
| content | Sample description | T = 0 | M | M | M | M | M |
| 2 mg/mL | 5 mM Citrate buffered, | 6.4 | 6.4 | 6.4 | | 6.5 | |
| | with Kolliphor ™ ELP | | | | | | |
| | 5 mM Citrate buffered, | 6.4 | 6.4 | 6.4 | 6.3 | 6.4 | 6.4 |
| | with Kolliphor ™ EL | | | | | | |
| | 2.5 mM Citrate buffered, | 6.5 | 6.4 | 6.5 | 6.6 | 6.6 | 6.8 |
| | with Kolliphor ™ EL | | | | | | |
| | Reference sample 1 | 6.2 | 6.2 | 6.1 | | 6.0 | 5.9 |
| 10 mg/mL | 5 mM Citrate buffered, | 6.3 | | 6.3 | 6.3 | 6.3 | 6.4 |
| | with Kolliphor ™ ELP | | | | | | |
| | 5 mM Citrate buffered, | 6.4 | 6.3 | 6.4 | 6.3 | 6.4 | 6.4 |
| | with Kolliphor ™ EL | | | | | | |
| | 2.5 mM Citrate buffered, | 6.5 | 6.5 | 6.5 | | 6.5 | 6.5 |
| | with Kolliphor ™ EL | | | | | | |
| | 1 mM Citrate buffered, | 6.2 | 6.3 | 6.3 | | 6.3 | 6.2 |
| | with Kolliphor ™ ELP | | | | | | |
| | Reference sample 2 | 6.4 | 6.3 | 6.3 | | 6.2 | 6.3 |

Independent of the type of Kolliphor™ used, the pH—for both Phytonadione concentrations (2 mg/mL and 5 mg/mL) did not shift by more than 0.3 pH units in any of the citrate buffer systems (1.0 mM, 2.5 mM and 5 mM).

### Headspace Oxygen

TABLE 2b
Headspace oxygen samples (all values in %)

| Phytonadione | | | 25° C./3 | 25° C./6 | 40° C./3 | 40° C./6 |
|---|---|---|---|---|---|---|
| content | Sample description | T = 0 | M | M | M | M |
| 2 mg/mL | 5 mM Citrate buffered, with | | | | | |
| | Kolliphor ™ ELP | | | | | |
| | 5 mM Citrate buffered, with | 2.508 | | 3.032 | 3.353 | 3.795 |
| | Kolliphor ™ EL | | | | | |
| | 2.5 mM Citrate buffered, with | 2.410 | | 2.990 | 3.124 | 3.668 |
| | Kolliphor ™ EL | | | | | |
| | Reference sample 1 | 1.020 | 1.860 | 0.830 | 1.702 | 0.753 |
| 10 mg/mL | 5 mM Citrate buffered, with | 2.473 | | 3.217 | 2.875 | 3.235 |
| | Kolliphor ™ ELP | | | | | |
| | 5 mM Citrate buffered, with | 2.428 | | 2.959 | 2.711 | 2.671 |
| | Kolliphor ™ EL | | | | | |
| | 2.5 mM Citrate buffered, with | 2.629 | | 2.730 | 2.431 | 1.986 |
| | Kolliphor ™ EL | | | | | |
| | 1 mM Citrate buffered, with | 2.212 | 2.787 | 3.180 | 3.032 | 2.909 |
| | Kolliphor ™ ELP | | | | | |
| | Reference sample 2 | 1.281 | 1.940 | 0.915 | 1.923 | 0.820 |

The headspace oxygen content in the citrate buffered samples in vials remained generally unchanged, or slightly increased over 6 months of storage. By contrast, the headspace oxygen content in the reference samples stored in ampules generally decreased over 6 months of storage.

### Phytonadione Content

TABLE 2c
API assay (% of theoretical phytonadione content)

| Phytonadione | | | 25° C./3 | 25° C./6 | 25° C./12 | 40° C./3 | 40° C./6 |
|---|---|---|---|---|---|---|---|
| content | Sample description | T = 0 | M | M | M | M | M |
| 2 mg/mL | 5 mM Citrate buffered, | 97.6 | 98.9 | 96.6 | | 96.6 | |
| | with Kolliphor ™ ELP | | | | | | |
| | 5 mM Citrate buffered, | 100.3 | 99.8 | 98.8 | 103.5 | 99.9 | 98.3 |
| | with Kolliphor ™ EL | | | | | | |
| | 2.5 mM Citrate buffered, | 100.6 | 100.2 | 97.0 | 102.9 | 99.3 | 97.4 |
| | with Kolliphor ™ EL | | | | | | |
| | Reference sample 1 | 100.2 | 100.1 | 99.4 | | 98.6 | 98.4 |
| 10 mg/mL | 5 mM Citrate buffered, | 102.3 | | 103.7 | 101.8 | 101.5 | 100.9 |
| | with Kolliphor ™ ELP | | | | | | |
| | 5 mM Citrate buffered, | 98.1 | 97.9 | 96.8 | 99.3 | 96.8 | 94.9 |
| | with Kolliphor ™ EL | | | | | | |
| | 2.5 mM Citrate buffered, | 98.4 | 98.3 | 96.2 | 99.1 | 96.5 | 94.0 |
| | with Kolliphor ™ EL | | | | | | |
| | 1 mM Citrate buffered, | 99.6 | 100.7 | 99.5 | | 99.7 | 95.8 |
| | with Kolliphor ™ ELP | | | | | | |
| | Reference sample 2 | 102.7 | 102.7 | 100.5 | 105.3 | 102.9 | 100.7 |

The API assay value of the citrate buffered samples in vials was comparable to the API assay value of the reference samples in ampoules over the investigated storage duration at both temperatures.

### Total Impurities

TABLE 2d
Total impurities (% of theoretical phytonadione content)

| Phytonadione | | | 25° C./3 | 25° C./6 | 25° C./12 | 40° C./3 | 40° C./6 |
|---|---|---|---|---|---|---|---|
| content | Sample description | T = 0 | M | M | M | M | M |
| 2 mg/mL | 5 mM Citrate buffered, | 0.97 | 0.29 | 0.49 | | 0.67 | |
| | with Kolliphor ™ ELP | | | | | | |
| | 5 mM Citrate buffered, | 0.86 | 0.61 | 1.53 | 1.23 | 0.88 | 1.52 |
| | with Kolliphor ™ EL | | | | | | |
| | 2.5 mM Citrate buffered, | 0.64 | 0.67 | 1.78 | 1.28 | 0.97 | 1.77 |
| | with Kolliphor ™ EL | | | | | | |
| | Reference sample 1 | 2.12 | 1.95 | 2.59 | | 2.09 | 3.22 |
| 10 mg/mL | 5 mM Citrate buffered, | 0.31 | | 0.31 | 0.57 | 0.75 | 1.29 |
| | with Kolliphor ™ ELP | | | | | | |
| | 5 mM Citrate buffered, | 0.45 | 0.67 | 0.98 | 1.47 | 1.23 | 2.06 |
| | with Kolliphor ™ EL | | | | | | |
| | 2.5 mM Citrate buffered, | 0.68 | 0.91 | 1.37 | 2.12 | 1.44 | 2.28 |
| | with Kolliphor ™ EL | | | | | | |
| | 1 mM Citrate buffered, | 0.13 | 0.17 | 0.62 | | 0.46 | 1.62 |
| | with Kolliphor ™ ELP | | | | | | |
| | Reference sample 2 | 2.83 | 2.64 | 4.41 | 3.40 | 2.87 | 3.89 |

The change in impurity content of the citrate buffered samples in vials over the storage duration was similar to the unbuffered reference samples in ampoules.

Overall, these results demonstrate the feasibility of citrate buffered phytonadione formulations according to the present invention comprising a polyethoxylated castor oil in the absence of antioxidants for both phytonadione concentrations—the citrate buffered samples show an at least comparable stability to the unbuffered reference samples stored in ampoules on the basis of the parameters described herein.

### Example 3: Comparison of Different Buffer Systems

Dispersions were prepared according to example 1 with a phytonadione content of 10 mg/mL and three different buffer systems: Citrate buffer pH=6.3, 5 mmol/L, phosphate buffer pH=6.3, 5 mmol/L and histidine buffer pH=6.3, 5 mmol/L. One aliquot of each of the dispersions was incubated for 12 hours in a stainless-steel tank to gain insight into the impact of Fe³⁺ ions on the stability of the samples, a second aliquot was incubated for 12 hours in a glass beaker without stainless-steel contact. Samples from both aliquots were filled in amber vials and stored in stability chambers as described in example 2.

PH-values, headspace oxygen and phytonadione content were investigated using the analytical methods described above.

### Results:

### pH Value

TABLE 3a
pH -values

| | | | 10 mg/mL-5 mM | | 10 mg/mL-5 mM | | 10 mg/mL-5 mM L- | |
|---|---|---|---|---|---|---|---|---|
| | | | Phosphate buffered | | Citrate buffered | | Histidine buffered | |
| | | | 12 h glass | 12 h steel | 12 h glass | 12 h steel | 12 h glass | 12 h steel |
| | | | contact | contact | contact | contact | contact | contact |
| t = 0 | | | 6.4 | 6.3 | 6.2 | 6.4 | 6.4 | 6.4 |
| 25° C. | 3 | months | 6.3 | 6.3 | 6.3 | 6.3 | 6.4 | 6.4 |
| | 6 | months | 6.3 | 6.3 | 6.3 | 6.3 | 6.4 | 6.4 |
| | 12 | months | 6.1 | 6.1 | 6.3 | 6.4 | 6.3 | 6.3 |
| 40° C. | 1 | month | | 6.2 | | 6.3 | | 6.3 |
| | 3 | months | 5.8 | 5.7 | 6.4 | 6.4 | 6.2 | 6.2 |
| | 6 | months | 5.2 | 5.2 | 6.4 | 6.5 | 6.1 | 6.1 |

The pH drift of the samples comprising citrate buffer did not shift at any of the storing conditions, whereas for the histidine- and phosphate buffered samples a pH-drop was observed.

### Headspace Oxygen

TABLE 3b
Headspace oxygen samples (all values in %)

| | | | 10 mg/mL-5 mM | | 10 mg/mL-5 mM | | 10 mg/mL-5 mM L- | |
|---|---|---|---|---|---|---|---|---|
| | | | Phosphate buffered | | Citrate buffered | | Histidine buffered | |
| | | | 12 h glass | 12 h steel | 12 h glass | 12 h steel | 12 h glass | 12 h steel |
| | | | contact | contact | contact | contact | contact | contact |
| 25° C. | 6 | months | 1.537 | 1.649 | 2.860 | 2.972 | 1.526 | 1.272 |
| 40° C. | 6 | months | 0.211 | 0.208 | 2.716 | 2.699 | 0.171 | 0.284 |
| 25° C. | 12 | months | 0.928 | 0.781 | 3.416 | 3.608 | 1.140 | 1.267 |

The headspace oxygen content of citrate buffered samples was generally unchanged or slightly increased over storage. There was a substantial decrease in the headspace oxygen content in the histidine buffered samples after 6 months at 40° C. and in the phosphate buffered samples after 6 months at 25° C. or 40° C.

### Phytonadione Content

TABLE 3c
API assay (% of theoretical phytonadione content)

| | | | 10 mg/mL-5 mM | | 10 mg/mL-5 mM | | 10 mg/mL-5 mM L- | |
|---|---|---|---|---|---|---|---|---|
| | | | Phosphate buffered | | Citrate buffered | | Histidine buffered | |
| | | | 12 h glass | 12 h steel | 12 h glass | 12 h steel | 12 h glass | 12 h steel |
| | | | contact | contact | contact | contact | contact | contact |
| T = 0 | | | 98.7 | 98.6 | 97.8 | 98.5 | 97.6 | 97.6 |
| 25° C. | 3 | months | 98.0 | 98.3 | 98.5 | 98.8 | 97.2 | 96.9 |
| | 6 | months | 99.1 | 96.4 | 99.9 | 101.1 | 98.3 | 98.3 |
| | 12 | months | 95.9 | 95.3 | 100.5 | 100.2 | 96.3 | 96.9 |
| 40° C. | 1 | month | | 98.3 | | 98.9 | | 97.6 |
| | 3 | months | 96.6 | 96.5 | 98.1 | 97.7 | 96.3 | 95.1 |
| | 6 | months | 96.6 | 96.5 | 98.5 | 98.8 | 96.5 | 95.8 |

The phytonadione content in the citrate buffered samples was generally unchanged over the storage duration, whereas both in histidine and in phosphate buffer systems a slight decrease of the phytonadione content was observed.
Without being bound by theory, it appears that the stabilizing properties of the citrate buffer cannot be explained by its iron complexing properties (i.e. by removing catalytically active metal ions from the solution), since there were no apparent differences between the citrate-buffered samples pre-incubated in a stainless-steel container and the samples pre-incubated in a glass container.

1-16. (canceled)

17. An aqueous pharmaceutical composition for parenteral administration to a human, comprising a pharmacologically effective amount of a pharmaceutically acceptable vitamin K, a polyethoxylated castor oil as solubilizer, and a citrate buffer at a concentration of 1 to 40 mmol/L, wherein the pH of the composition is between 3.5 and 7.0.

18. The composition according to claim 17, wherein the composition comprises citrate buffer at a concentration of 1 to 20 mmol/L.

19. The composition according to claim 17, wherein the pH of the composition is between 5.0 and 7.0.

20. The composition according to claim 17, wherein the polyethoxylated castor oil is obtained by reacting castor oil with ethylene oxide in a molar ratio of 1:35.

21. The composition according to claim 20, wherein the composition comprises 30 to 150 mg macrogolglycerol ricinoleate polyoxyl-35-castor oil per mL of the composition.

22. The composition according to claim 17, wherein the pharmaceutically acceptable vitamin K is vitamin K₁ (phytonadione).

23. The composition according to claim 22, comprising 0.1 to 50 mg phytonadione per mL of the composition.

24. The composition according to claim 23, comprising 2 mg phytonadione per mL of the composition or 10 mg phytonadione per mL of the composition.

25. The composition of claim 24, comprising 1-10 mmol/L citrate buffer and having a pH between 6.0 and 6.5.

26. The composition of claim 25, wherein the pH drift of the composition is less than 0.2 pH units following storage for 3 months at 40° C. or for 12 months at 25° C.

27. The composition of claim 25, wherein the composition contains 97% to 103% of the theoretical amount of phytonadione following storage for 3 months at 40° C. or for 12 months at 25° C.

28. The composition according to claim 17, wherein the composition does not comprise butylated hydroxyanisole or butylated hydroxytoluene.

29. The composition according to claim 22, comprising phytonadione, citrate buffer at a concentration of 1 to 20 mmol/L, dextrose, benzyl alcohol, 30 to 150 mg macrogolglycerol ricinoleate polyoxyl-35-castor oil per mL of the composition, water for injection and glacial acetic acid, wherein the composition has a pH between 6.0 and 6.5.

30. The composition according to claim 29, comprising 2 mg phytonadione per mL of the composition, 5 mmol citrate buffer per liter of the composition, 37.5 mg dextrose monohydrate per mL of the composition, 9 mg benzyl alcohol per mL of the composition, and 70 mg macrogolglycerol ricinoleate polyoxyl-35-castor oil per mL of the composition, wherein the composition has a pH of 6.3.

31. The composition of claim 30, wherein following storage for 3 months at 40° C. or for 12 months at 25° C., the pH drift of the composition is less than 0.2 pH units and the composition contains 97% to 103% of the theoretical amount of phytonadione.

32. The composition according to claim 29, comprising 10 mg phytonadione per mL of the composition, 5 mmol citrate buffer per liter of the composition, 37.5 mg dextrose monohydrate per mL of the composition, 9 mg benzyl alcohol per mL of the composition, and 70 mg macrogolglycerol ricinoleate polyoxyl-35-castor oil per mL of the composition, wherein the pharmaceutical composition has a pH of 6.3.

33. The composition of claim 32, wherein following storage for 3 months at 40° C. or for 12 months at 25° C., the pH drift of the composition is less than 0.2 pH units and the composition contains 97% to 103% of the theoretical amount of phytonadione.

34. A pharmaceutically acceptable container comprising a pharmaceutical composition according to claim 17, wherein the container is not an ampoule.

35. The container according to claim 34, wherein the container is a vial sealed with a stopper, a syringe or a cartridge.

36. A method for the treatment or prevention of vitamin K deficiency and/or haemorrhage or for use as an antidote to anticoagulants of the coumarin type in a human, comprising administering the composition according to claim 17 to a human in need thereof.
